(19) United States
(12) Patent Application Publication
(10) Pub. No.: US 2026/0229311 A1
(43) Pub. Date: Aug. 6, 2026

(54) METHOD TO DISCOVER PUTATIVE GENES IN SINGLE CELL ANALYSIS

(71) Applicant: INTERNATIONAL BUSINESS MACHINES CORPORATION, Armonk, NY (US)

(72) Inventors: Uri Kartoun, Cambridge, MA (US); Akira Koseki, Yokohama-shi (JP); Natalia Mulligan, Dublin (IE); Joao H. Bettencourt-Silva, Dublin (IE); Vibha Anand, Chestnut Hill, MA (US)

(21) Appl. No.: 19/044,875

(22) Filed: Feb. 4, 2025

(51) Int. Cl. G16B 30/00; G16B 40/20

(52) U.S. Cl. CPC G16B 30/00; G16B 40/20

(57) ABSTRACT

Methods of, systems of, and computer program products for analyzing gene expression data (e.g., single-cell RNA sequencing data) for the identification of patterns within cell types are provided. In some embodiments, methods of, systems of, and computer products for ranking genes based on patterns of gene expression within a cell type are provided. The method can employ three or more foundation models to determine differences those model's analyses of gene expression.

### BACKGROUND

The global single-cell analysis market is expected to have over an 18% compound annual growth rate over the next five years. This growth is driven by the expanding use of single-cell gene expression data in disease diagnosis, drug discovery, and drug development. While technological innovations in single-cell isolation and sequencing are advancing, interpretation of this data is lagging due to variations in datasets and model interpretability.

### BRIEF SUMMARY

Embodiments of the present disclosure relate to use of computational models for analyzing gene expression data for the identification of patterns within cell types, and more specifically, to the use of computation models in analyzing single-cell ribonucleic acid (RNA) sequencing data in accelerating target discovery for drug development.

According to embodiments of the present disclosure, methods of, systems of, and computer program products for analyzing gene expression data (e.g., single-cell RNA sequencing data) are provided. In some embodiments, methods of, systems of, and computer products for ranking genes based on patterns of gene expression within a cell type are provided. In some embodiments, methods of, systems of, and computer products for ranking genes based on their potential as a druggable target are provided.

In one embodiment, a method comprises loading at least three foundation models, each of the models trained on single-cell RNA sequencing (scRNA-seq) data. The method can comprise loading a domain-specific dataset, the domain-specific dataset being a subset of scRNA-seq data. The method can comprise generating, for each of the at least three foundation models, a fine-tuned model associated with that model by training that model using the domain-specific dataset. The method can comprise providing, to each of the at least three foundation models, single-cell data, the single-cell data associated with a cell type and a plurality of genes associated with that cell type. The method can comprise receiving, from each of the at least three of foundation models, a plurality of foundation model attribution scores, each foundation model attribution score being associated with one of the genes of the single-cell data. The method can comprise providing, to each of the at least three of fine-tuned models, the single-cell data. The method can comprise receiving, from each of the at least three of fine-tuned models, a fine-tuned model attribution score, each fine-tuned model attribution score being associated with one of the genes of the single-cell data. The method can comprise calculating, for each of the at least three foundation models, a plurality of ratio scores, each ratio score being associated with one of the plurality of genes, each ratio score being a ratio of the foundation model attribution score received from that foundation model and for that gene and the fine-tuned model attribution score received from the fine-tuned model associated with that foundation model and for that gene. The method can comprise calculating, for each pair of foundation models in the plurality of foundation models, a plurality of a deviation metrics, each deviation metric being associated with one of the plurality of genes, each deviation metric representing a deviation in attribution scores between a first foundation model and a second foundation model of that pair. The method can comprise selecting at least one deviation score that is above a threshold. The method can comprise outputting at least one gene, the at least one gene being associated with the selected at least one deviation score.

In some embodiments, the foundation models are trained on unlabeled data. In some embodiments, the at least one deviation score is one or more of a standard deviation, a MAX−MIN, Q3−Q1, average mean absolute deviation (MAD), and coefficient of variation. In some embodiments, the threshold is configured to select a top scoring percentage of the genes associated with the single-cell data. In some embodiments, at least one of the plurality of foundation models is a pre-trained model. In some embodiments, the at least one gene is a putative gene.

In some embodiments, the method further comprises loading a database including information about the single-cell data. The method can comprise extracting, using a natural language processing model, a consistency metric for at least one gene of the single-cell data. The method can comprise determining, based on the consistency metric, the threshold.

In some embodiments, the method further comprises loading single-cell scRNA-seq data from a database. The method can comprise training a first foundation model of the plurality of foundation models using the single-cell scRNA-seq data and a first plurality of parameters. The method can comprise training a second foundation model of the plurality of foundation models using the single-cell scRNA-seq data and a second plurality of parameters. The method can comprise wherein the first plurality of parameters is different from the second plurality of parameters.

In some embodiments, a systems comprises a computing node comprising a computer readable storage medium having program instructions embodied therewith. The program instructions can be executable by a processor of the computing node to cause the processor to perform a method. The method executable by the processor comprising loading at least three foundation models, each of the models trained on single-cell RNA sequencing (scRNA-seq) data. The method can comprise loading a domain-specific dataset, the domain-specific dataset being a subset of scRNA-seq data. The method can comprise generating, for each of the at least three foundation models, a fine-tuned model associated with that model by training that model using the domain-specific dataset. The method can comprise providing, to each of the at least three foundation models, single-cell data, the single-cell data associated with a cell type and a plurality of genes associated with that cell type. The method can comprise receiving, from each of the at least three of foundation models, a plurality of foundation model attribution scores, each foundation model attribution score being associated with one of the genes of the single-cell data. The method can comprise providing, to each of the at least three of fine-tuned models, the single-cell data. The method can comprise receiving, from each of the at least three of fine-tuned models, a fine-tuned model attribution score, each fine-tuned model attribution score being associated with one of the genes of the single-cell data. The method can comprise calculating, for each of the at least three foundation models, a plurality of ratio scores, each ratio score being associated with one of the plurality of genes, each ratio score being a ratio of the foundation model attribution score received from that foundation model and for that gene and the fine-tuned model attribution score received from the fine-tuned model associated with that foundation model and for that gene. The method can comprise calculating, for each pair of foundation models in the plurality of foundation models, a plurality of a deviation metrics, each deviation metric being associated with one of the plurality of genes, each deviation metric representing a deviation in attribution scores between a first foundation model and a second foundation model of that pair. The method can comprise selecting at least one deviation score that is above a threshold. The method can comprise outputting at least one gene, the at least one gene being associated with the selected at least one deviation score.

In some embodiments, the foundation models are trained on unlabeled data. In some embodiments, the at least one deviation score is one or more of a standard deviation, a MAX−MIN, Q3−Q1, average mean absolute deviation (MAD), and coefficient of variation. In some embodiments, the threshold is configured to select a top scoring percentage of the genes associated with the single-cell data. In some embodiments, at least one of the plurality of foundation models is a pre-trained model.

In some embodiments, the method performed by the processor further comprises loading a database including information about the single-cell data. The method can comprise extracting, using a natural language processing model, a consistency metric for at least one gene of the single-cell data. The method can comprise determining, based on the consistency metric, the threshold.

In some embodiments, the method performed by the process further comprises loading single-cell scRNA-seq data from a database. The method can comprise training a first foundation model of the plurality of foundation models using the single-cell scRNA-seq data and a first plurality of parameters. The method can comprise training a second foundation model of the plurality of foundation models using the single-cell scRNA-seq data and a second plurality of parameters. The method can comprise wherein the first plurality of parameters is different from the second plurality of parameters.

In some embodiments, a computer program product for selecting genes comprises a computer readable storage medium having program instructions embodied therewith. The program instructions can be executable by a processor to cause the processor to perform a method comprising loading at least three foundation models. Each of the models can be trained on single-cell RNA sequencing (scRNA-seq) data. The method can comprise loading a domain-specific dataset, the domain-specific dataset being a subset of scRNA-seq data. The method can comprise generating, for each of the at least three foundation models, a fine-tuned model associated with that model by training that model using the domain-specific dataset. The method can comprise providing, to each of the at least three foundation models, single-cell data, the single-cell data associated with a cell type and a plurality of genes associated with that cell type. The method can comprise receiving, from each of the at least three of foundation models, a plurality of foundation model attribution scores, each foundation model attribution score being associated with one of the genes of the single-cell data. The method can comprise providing, to each of the at least three of fine-tuned models, the single-cell data. The method can comprise receiving, from each of the at least three of fine-tuned models, a fine-tuned model attribution score, each fine-tuned model attribution score being associated with one of the genes of the single-cell data. The method can comprise calculating, for each of the at least three foundation models, a plurality of ratio scores, each ratio score being associated with one of the plurality of genes, each ratio score being a ratio of the foundation model attribution score received from that foundation model and for that gene and the fine-tuned model attribution score received from the fine-tuned model associated with that foundation model and for that gene. The method can comprise calculating, for each pair of foundation models in the plurality of foundation models, a plurality of a deviation metrics, each deviation metric being associated with one of the plurality of genes, each deviation metric representing a deviation in attribution scores between a first foundation model and a second foundation model of that pair. The method can comprise selecting at least one deviation score that is above a threshold. The method can comprise outputting at least one gene, the at least one gene being associated with the selected at least one deviation score.

In some embodiments, the foundation models are trained on unlabeled data. In some embodiments, the at least one deviation score is one or more of a standard deviation, a MAX−MIN, Q3−Q1, average mean absolute deviation (MAD), and coefficient of variation. In some embodiments, the threshold is configured to select a top scoring percentage of the genes associated with the single-cell data.

In some embodiments, the method performed by the processor further comprises loading a database including information about the single-cell data. The method can comprise extracting, using a natural language processing model, a consistency metric for at least one gene of the single-cell data. The method can comprise determining, based on the consistency metric, the threshold.

### BRIEF DESCRIPTION OF THE DRAWINGS

FIG. 1 is a flowchart illustrating identifying genes with high probability for being successful targets for drug discovery using large databases of cell-related data according to embodiments of the present disclosure.

FIG. 2A is a graph illustrating attribution scores of genes in an exemplary analysis of single-cell RNA sequencing data from goblet cells from the pre-trained and fine-tuned models according to embodiments of the present disclosure.

FIG. 2B is a graph illustrating ratios of the attribution scores of genes as illustrated by FIG. 2A according to embodiments of the present disclosure.

FIG. 3 is a block diagram illustrating generating a set of genes with expression for a given cell type that can be considered as targets for drug discovery according to embodiments of the present disclosure.

FIG. 4A-B are tables, respectively, illustrating example results (e.g., ratios of attribution scores) of the method according to embodiments of the present disclosure.

FIG. 5 is a diagram illustrating yea computing node according to an embodiment of the present disclosure.

### DETAILED DESCRIPTION

Single-cell sequencing is a molecular biology technique that enables the analysis of the genomic, transcriptomic, or epigenomic characteristics of individual cells, rather than bulk tissue samples. This method provides high-resolution insights into cellular heterogeneity, allowing the detection of rare cell types, genetic mutations, and subtle variations in gene expression that would otherwise be masked in population-based analyses. The technique typically involves isolating individual cells, followed by amplification of their genetic material, sequencing, and data analysis to reconstruct the molecular profile of each cell. Single-cell sequencing has broad applications in fields such as cancer research, developmental biology, immunology, and disease modeling, providing a powerful tool for understanding complex biological processes at the single-cell level. However, single-cell sequencing data can be challenging to interpret due to the inherent noise and technical variability introduced during sample preparation and sequencing, necessitating the development of computational processes for data processing and interpretation.

Described herein are methods of, systems of, and computer program products for analyzing gene expression data (e.g., single-cell RNA sequencing data). In some embodiments, methods of, systems of, and computer products for ranking genes based on patterns of gene expression within a cell type are provided. In some embodiments, methods of, systems of, and computer products for ranking genes based on their potential as a druggable target are provided. The results of such methods can be used to inform drug development, such as in the design of perturbation experiments, in the design and construction of gene regulatory networks, and in research into cell signaling pathways.

In some embodiments described herein, foundation models (FMs) are used in the interpretation of gene expression data. In some embodiments, foundation models are trained on gene expression data (e.g., single-cell RNA sequencing data). In some embodiments, multiple foundation models are each trained on a unique set of gene expression data (e.g., single-cell RNA sequencing data). In some embodiments, the outputs generated by multiple foundation models are input into an interpretation model. In some embodiments, the interpretation model identifies a set of genes. In some embodiments, the interpretation model identifies a subset of putative genes. In some embodiments, the identified subset of genes can be used to assess potentially novel genetic associations in a single-cell analysis. In some embodiments, the identified subset of genes can be used to assess the level of consistency of the potentially novel genetic associations using multiple independent models. In some embodiments, the identified subset of genes can be used to identified models that hold a discrepancy compared to other models within the context of the potentially novel genetic associations.

In some embodiments, single-cell sequencing includes examining nucleic acid sequence information (e.g., DNA, RNA) from individual cells with sequencing technologies (e.g., next-generation sequencing), thereby providing a higher resolution of cellular differences and a better understanding of the function of an individual cell in the context of its microenvironment. As one example, in cancer, sequencing of DNA of individual cells can give information about mutations carried by small populations of cells. In some development examples, sequencing RNAs expressed by individual cells can give insight into the existence and behavior of different cell types. In microbial systems, a population of the same species can appear genetically clonal. In some embodiments, single-cell sequencing of RNA or epigenetic modifications can reveal cell-to-cell variability that may help populations rapidly adapt to survive in changing environments.

In some embodiments, a putative gene is a DNA segment that is believed to be a gene, but its function is unknown. In some embodiments, the putative gene can be considered a candidate gene.

In some embodiments, a foundational model, also known as a large artificial intelligence (AI) model, can be a machine learning or deep learning model that is trained on broad data such that it can be applied across a wide range of use cases. In some embodiments, the foundation models described herein are based deep neural networks. In some embodiments, the foundation models are trained using unsupervised or self-supervised learning.

In some embodiments, the foundation models can be pretrained. Pre-training the foundation model comprises training a large neural network on a broad or vast dataset. Such a dataset can be diverse and not specific to a particular task. In such instances, the model learns a broad understanding of the data and develops a generalized capability to understand input data and can make reasonable predictions or representations of that data.

In some embodiments, the foundation models can be fine-tuned. After a model has been pre-trained, it can be fine-tuned on a smaller, more specific dataset. In such instances, the weights of the model are adjusted to perform well on a particular task. Such a process can comprise continuing the training process of the pre-trained model.

In some embodiments described herein, interpretation models are used. In some embodiments, Captum, an interpretability library is used as the basis of an interpretation model. In some embodiments, the interpretation model offers feature attribution (i.e., explanation of how individual input features contribute to the model's predictions). In some embodiments, Local Interpretable Model-agnostic Explanations (LIME) is method that generates interpretable models locally around each prediction to explain individual predictions by approximating the black-box model with a simpler model. In some embodiments, Pre-training SHAP (SHapley Additive exPlanations) can provide a measure of feature importance by assigning each feature a value based on its contribution to a model's prediction, using concepts from game theory.

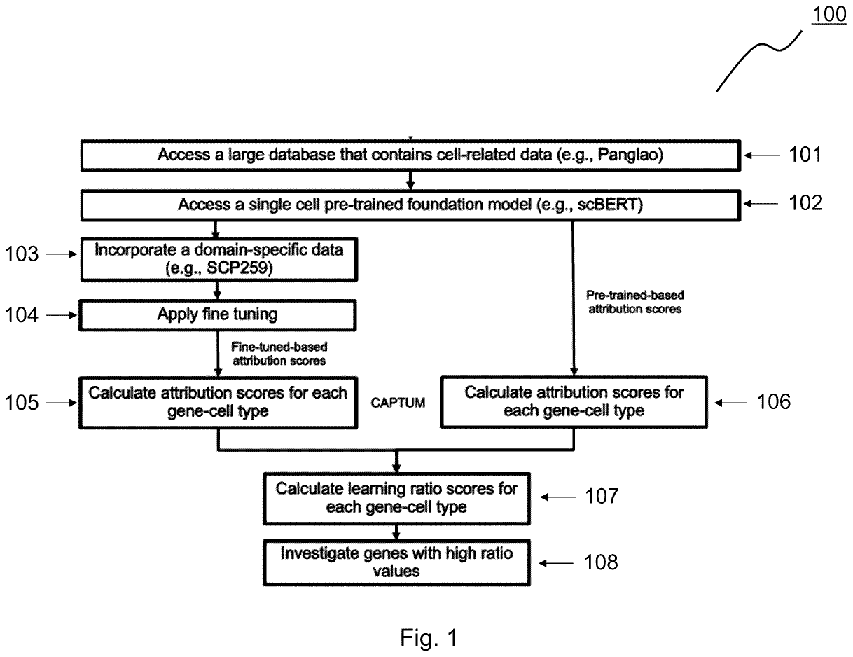

FIG. 1 is a flowchart 100 illustrating identifying genes with high probability for being successful targets for drug discovery using large databases of cell-related data according to embodiments of the present disclosure. First, a large database that contains cell-related data is accessed (101). Examples of such a database include PanglaoDB, an open-access database for single-cell RNA sequencing data. The method can access a single cell pre-trained (e.g., on the database from step 101) foundation model is accessed (102). An example of such a model include scBERT, which is a large-scale pretrained deep language model for cell type annotation of single-cell RNA-seq data. The method can then perform two sub-processes (e.g., 103-105 and 106) in parallel. In one sub-process, domain-specific data can be accessed (103). In one embodiment, the domain-specific data can include a domain such as SCP259, which is a domain of genes that relate to intra-and inter-cellular rewiring of the human colon during ulcerative colitis. However, other domains can be used for fine-tuning. Then, the single cell pre-trained foundation model can be fine-tuned using the domain-specific data (104), thereby providing a fine-tuned model. This fine-tuned model can then generate fine-tune-based attribution scores for each gene-cell type using that domain-specific model (105). In some embodiments, an attribution score is a numerical value that represents a strength or a correlation measure that a given gene belongs to a cell-type of interest. In some embodiments, the measure could be a probability or likelihood. In the other sub-process, the single cell pre-trained foundation model (e.g., scBERT) can calculate attribution scores for each gene-cell type (106). In some embodiments, the attribution scores can be calculated using a layer integrated gradients method, such as Captum. The attribution scores from each track can be compared and used to calculate a learning ratio score for each gene-cell type (107). Then, genes with high ratio values can be further investigated for their potential as druggable targets (108).

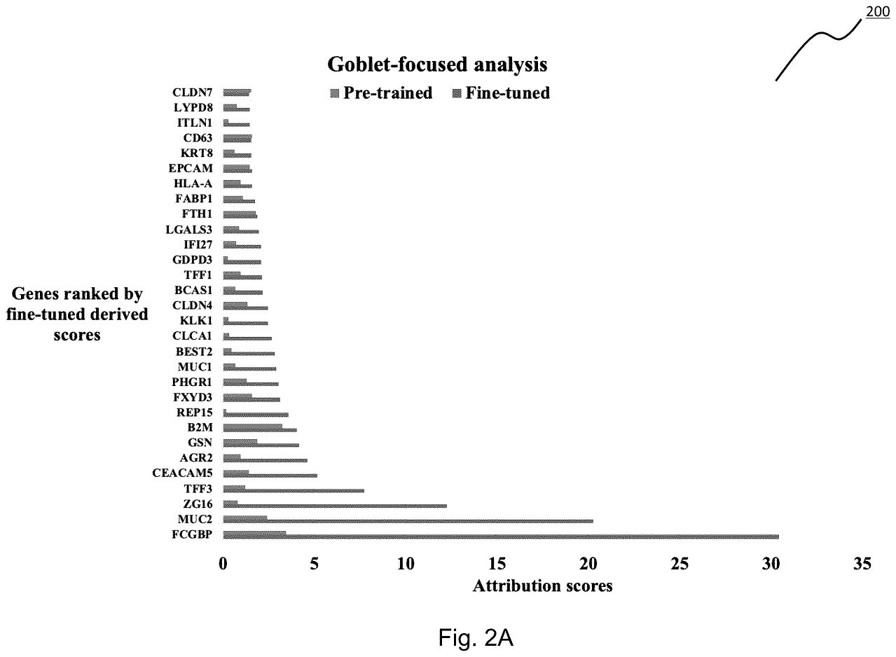

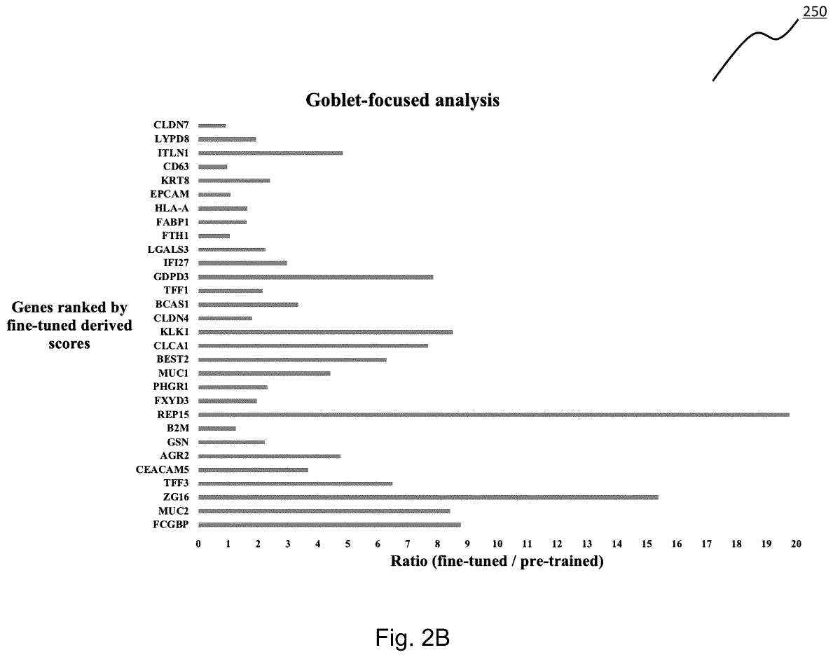

In some embodiments, the method comprises pre-training a foundation model for target discovery (referred to as BMFM-target). In an example of the process described in FIG. 1, a BMFM-target was trained using scRNA-seq data of over 1 million cells from PanglaoDB using a method similar to scBERT. It was then fine-tuned using a dataset from human colon mucosa of 18 ulcerative colitis and 12 healthy subjects (SCP259, ~6,000 genes and 51 cell types) from Smillie et al. “Intra-and Inter-cellular rewiring of the human colon during ulcerative colitis.” Cell (2019). A layer integrated gradients algorithm from Captum, an interpretation model, was then applied to calculate attribution scores of genes for predicting a cell type. The interpretation model was applied, for each gene-type, once for the pre-trained model and once for each fine-tuned model. A ratio, or a learning ratio, of the attribution scores was calculated by the interpretation model using the fine-tuned model to the attribution scores calculated by the interpretation model using the pre-trained model. The ratio thereby can represent a quantification of transfer learning and can highlight gene-cell type pairs to identify potentially important genes for the cell type. The results, shown by FIG. 2A and FIG. 2B and described further below, demonstrate that the fine-tuning model yielded higher attribution scores compared to the pre-training model.

FIG. 2A is a graph 200 illustrating attribution scores of genes in an exemplary analysis of single-cell RNA sequencing data from goblet cells from the pre-trained and fine-tuned models according to embodiments of the present disclosure. FIG. 2B is a graph 250 illustrating ratios of the attribution scores of genes as illustrated by FIG. 2A according to embodiments of the present disclosure.

High learning ratios for known genes were observed; for instance, for goblet cells, which produce mucus in the gut, the genes FCGBP and MUC2 had high attribution scores. For example, FCGBP is an important cell surface protein in gut cells, and MUC2 is a protein that forms mucus. Additionally, the process gave a learning ratio to REP15 (e.g., RAB15 effector protein) because it had a low score in the fine-tuned model but a high score in the pre-trained model, indicating that it may be an important gene for cellular dysfunction in the colon.

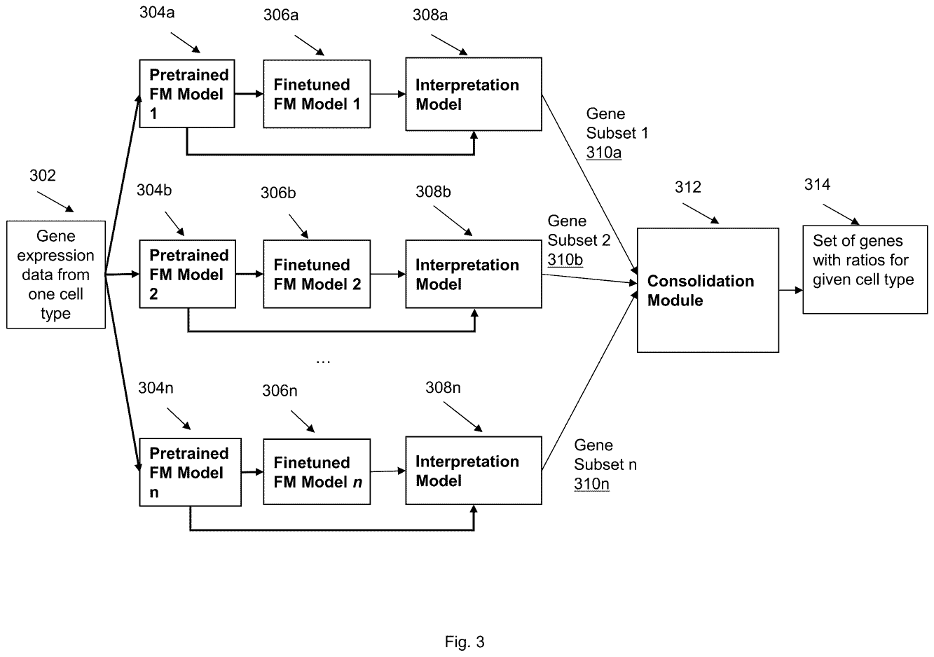

FIG. 3 is a block diagram 300 illustrating generating a set of genes with expression for a given cell type that can be considered as targets for drug discovery according to embodiments of the present disclosure. A gene expression dataset from one cell type 302 (e.g., from raw single cell RNA sequencing data) is provided to each pre-trained foundation model (FM Models) 304a-n. Each pre-trained foundational model 304a-n can be trained using different settings, parameters, hyperparameters, etc. It can be appreciated that in some embodiments, each foundational model 304a-n is trained on a same general database of gene expression data. Each of the pre-trained FM Models 304a-n is fine-tuned using the gene expression data 302, thereby resulting in finetuned FM Models 1-n 306a-n. An interpretation model 308 can then generate attribution scores of genes based on any one of the FM models. In particular, the interpretation model can generate attribution scores for a set of genes based on the pre-trained FM Model 304 and each of the finetuned FM Models 306a-n. The interpretation model can then calculate a ratio of the attribution scores of each gene generated by the pretrained FM Model 304a-n its corresponding finetuned FM model 306a-n. The interpretation model 308 can then select gene subsets 310a-n that have attribution scores above a particular threshold and output those subsets. Those gene subsets 310a-n are then provided to a consolidation module 312, which then outputs a set of genes 314 with their associated ratio for the particular cell type.

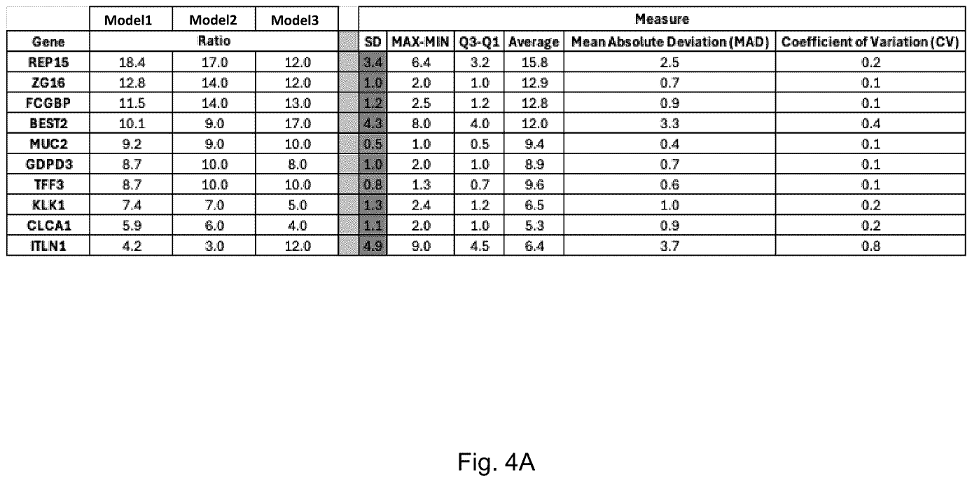

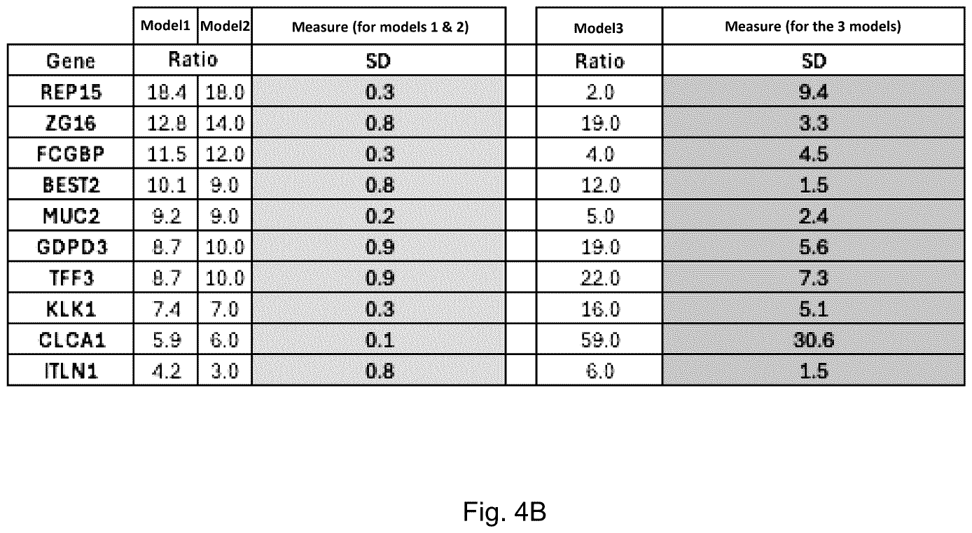

FIG. 4A and FIG. 4B are tables 400 and 450, respectively, illustrating example results (e.g., ratios of attribution scores) of the method according to embodiments of the present disclosure. In an example of the process as depicted in FIG. 1 and FIG. 3, three sets of gene expression data from goblet cells were analyzed by three foundation models and each interpreted by the same interpretation model to generate a learning ratio. “Ratios” can be based on comparisons of attribution scores for each genes generated by the fine-tuned model and the pre-trained models. By comparing the metric, such as a standard deviation, between the learning ratio for each, the consistency of genes can be observed. In the given example based on single-cell goblet cell data, MUC2 and TFF3 show a strong consistency across the three models. In contrast, REP15, BEST2, and ITLN1 have high variation (FIG. 4A). Notably, much of the variation is due to one particular model (model 3), while two of the models are closely aligned (model 1 and model 2) with similar learning ratios (FIG. 4B). Therefore, it can be recognized that analyzing a wide range of datasets through individual models can introduce needed variability into the gene set interpretation.

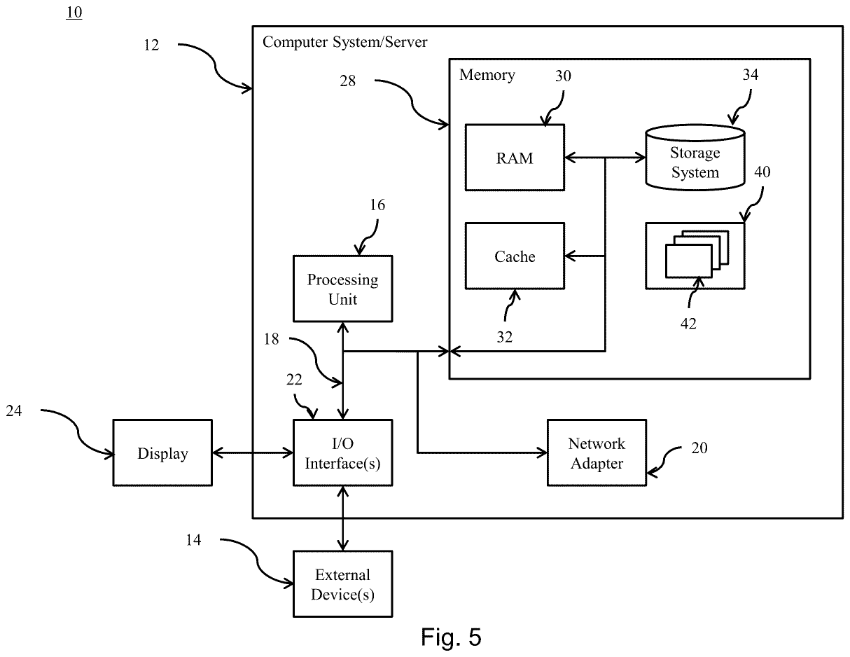

Referring now to FIG. 5, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
loading at least three foundation models, each of the models trained on single-cell RNA sequencing (scRNA-seq) data;
loading a domain-specific dataset, the domain-specific dataset being a subset of scRNA-seq data;
generating, for each of the at least three foundation models, a fine-tuned model associated with that model by training that model using the domain-specific dataset;
providing, to each of the at least three foundation models, single-cell data, the single-cell data associated with a cell type and a plurality of genes associated with that cell type;
receiving, from each of the at least three of foundation models, a plurality of foundation model attribution scores, each foundation model attribution score being associated with one of the genes of the single-cell data;
providing, to each of the at least three of fine-tuned models, the single-cell data;
receiving, from each of the at least three of fine-tuned models, a fine-tuned model attribution score, each fine-tuned model attribution score being associated with one of the genes of the single-cell data;
calculating, for each of the at least three foundation models, a plurality of ratio scores, each ratio score being associated with one of the plurality of genes, each ratio score being a ratio of the foundation model attribution score received from that foundation model and for that gene and the fine-tuned model attribution score received from the fine-tuned model associated with that foundation model and for that gene;
calculating, for each pair of foundation models in the plurality of foundation models, a plurality of a deviation metrics, each deviation metric being associated with one of the plurality of genes, each deviation metric representing a deviation in attribution scores between a first foundation model and a second foundation model of that pair;
selecting at least one deviation score that is above a threshold; and
outputting at least one gene, the at least one gene being associated with the selected at least one deviation score.

2. The method of claim 1, wherein the foundation models are trained on unlabeled data.

3. The method of claim 1, wherein the at least one deviation score is one or more of a standard deviation, a MAX−MIN, Q3−Q1, average mean absolute deviation (MAD), and coefficient of variation.

4. The method of claim 1, wherein the threshold is configured to select a top scoring percentage of the genes associated with the single-cell data.

5. The method of claim 1, further comprising:
loading a database including information about the single-cell data;
extracting, using a natural language processing model, a consistency metric for at least one gene of the single-cell data;
determining, based on the consistency metric, the threshold.

6. The method of claim 1, wherein at least one of the plurality of foundation models is a pre-trained model.

7. The method of claim 1, further comprising:
loading single-cell scRNA-seq data from a database;
training a first foundation model of the plurality of foundation models using the single-cell scRNA-seq data and a first plurality of parameters;
training a second foundation model of the plurality of foundation models using the single-cell scRNA-seq data and a second plurality of parameters;
wherein the first plurality of parameters is different from the second plurality of parameters.

8. The method of claim 1, wherein the at least one gene is a putative gene.

9. A system comprising:
a computing node comprising a computer readable storage medium having program instructions embodied therewith, the program instructions executable by a processor of the computing node to cause the processor to perform a method comprising:
loading at least three foundation models, each of the models trained on single-cell RNA sequencing (scRNA-seq) data;
loading a domain-specific dataset, the domain-specific dataset being a subset of scRNA-seq data;
generating, for each of the at least three foundation models, a fine-tuned model associated with that model by training that model using the domain-specific dataset;
providing, to each of the at least three foundation models, single-cell data, the single-cell data associated with a cell type and a plurality of genes associated with that cell type;
receiving, from each of the at least three of foundation models, a plurality of foundation model attribution scores, each foundation model attribution score being associated with one of the genes of the single-cell data;
providing, to each of the at least three of fine-tuned models, the single-cell data;
receiving, from each of the at least three of fine-tuned models, a fine-tuned model attribution score, each fine-tuned model attribution score being associated with one of the genes of the single-cell data;
calculating, for each of the at least three foundation models, a plurality of ratio scores, each ratio score being associated with one of the plurality of genes, each ratio score being a ratio of the foundation model attribution score received from that foundation model and for that gene and the fine-tuned model attribution score received from the fine-tuned model associated with that foundation model and for that gene;
calculating, for each pair of foundation models in the plurality of foundation models, a plurality of a deviation metrics, each deviation metric being associated with one of the plurality of genes, each deviation metric representing a deviation in attribution scores between a first foundation model and a second foundation model of that pair;
selecting at least one deviation score that is above a threshold; and
outputting at least one gene, the at least one gene being associated with the selected at least one deviation score.

10. The system of claim 9, wherein the foundation models are trained on unlabeled data.

11. The system of claim 9, wherein the at least one deviation score is one or more of a standard deviation, a MAX−MIN, Q3−Q1, average mean absolute deviation (MAD), and coefficient of variation.

12. The system of claim 9, wherein the threshold is configured to select a top scoring percentage of the genes associated with the single-cell data.

13. The system of claim 9, wherein the method performed by the processor further comprises:
loading a database including information about the single-cell data;
extracting, using a natural language processing model, a consistency metric for at least one gene of the single-cell data;
determining, based on the consistency metric, the threshold.

14. The system of claim 9, wherein at least one of the plurality of foundation models is a pre-trained model.

15. The system of claim 9, wherein the method performed by the process further comprises:
loading single-cell scRNA-seq data from a database;
training a first foundation model of the plurality of foundation models using the single-cell scRNA-seq data and a first plurality of parameters;
training a second foundation model of the plurality of foundation models using the single-cell scRNA-seq data and a second plurality of parameters;
wherein the first plurality of parameters is different from the second plurality of parameters.

16. A computer program product for selecting genes, the computer program product comprising a computer readable storage medium having program instructions embodied therewith, the program instructions executable by a processor to cause the processor to perform a method comprising:
loading at least three foundation models, each of the models trained on single-cell RNA sequencing (scRNA-seq) data;
loading a domain-specific dataset, the domain-specific dataset being a subset of scRNA-seq data;
generating, for each of the at least three foundation models, a fine-tuned model associated with that model by training that model using the domain-specific dataset;
providing, to each of the at least three foundation models, single-cell data, the single-cell data associated with a cell type and a plurality of genes associated with that cell type;
receiving, from each of the at least three of foundation models, a plurality of foundation model attribution scores, each foundation model attribution score being associated with one of the genes of the single-cell data;
providing, to each of the at least three of fine-tuned models, the single-cell data;
receiving, from each of the at least three of fine-tuned models, a fine-tuned model attribution score, each fine-tuned model attribution score being associated with one of the genes of the single-cell data;
calculating, for each of the at least three foundation models, a plurality of ratio scores, each ratio score being associated with one of the plurality of genes, each ratio score being a ratio of the foundation model attribution score received from that foundation model and for that gene and the fine-tuned model attribution score received from the fine-tuned model associated with that foundation model and for that gene;
calculating, for each pair of foundation models in the plurality of foundation models, a plurality of a deviation metrics, each deviation metric being associated with one of the plurality of genes, each deviation metric representing a deviation in attribution scores between a first foundation model and a second foundation model of that pair;
selecting at least one deviation score that is above a threshold; and
outputting at least one gene, the at least one gene being associated with the selected at least one deviation score.

17. The computer program product of claim 16, wherein the foundation models are trained on unlabeled data.

18. The computer program product of claim 16, wherein the at least one deviation score is one or more of a standard deviation, a MAX−MIN, Q3−Q1, average mean absolute deviation (MAD), and coefficient of variation.

19. The computer program product of claim 16, wherein the threshold is configured to select a top scoring percentage of the genes associated with the single-cell data.

20. The computer program product of claim 16, wherein the method performed by the processor further comprises:
loading a database including information about the single-cell data;
extracting, using a natural language processing model, a consistency metric for at least one gene of the single-cell data;
determining, based on the consistency metric, the threshold.
